(19) United States
(12) Patent Application Publication
(10) Pub. No.: US 2012/0130254 A1
(43) Pub. Date: May 24, 2012

(54) Dental Device for Trans-illumination of Teeth

(75) Inventors: André Hackel, Biberach (DE); Sven Erdmann, Ulm (DE)

(73) Assignee: Kaltenbach & Voigt GmbH, Biberach (DE)

(21) Appl. No.: 13/291,762

(22) Filed: Nov. 8, 2011

(30) Foreign Application Priority Data
Nov. 11, 2010 (DE) 10 2010 043 796.4

(51) Int. Cl. A61B 6/00

(52) U.S. Cl. 600/476

(57) ABSTRACT

In a dental system for the transillumination of teeth having an elongate handpiece with a light source for producing examination radiation, irradiation means for directing the examination radiation at a tooth to be examined, and means for acquiring an optical image of the tooth illuminated by the examination radiation, there is arranged at the front end of the handpiece a removable attachment which contains at least part of the irradiation means, the handpiece having means for detecting the attachment arranged on the handpiece.

The present invention relates to a dental system for the transillumination of teeth according to the precharacterising clause of claim 1, which system comprises an elongate handpiece with a light source for producing examination radiation, irradiation means for directing the examination radiation at a tooth to be examined, and means for acquiring an optical image of the tooth illuminated by the examination radiation, wherein there is arranged at the front end of the handpiece a removable attachment which contains at least part of the irradiation means.

In medicine, in particular in dentistry, diagnostic systems based on optical principles are increasingly being used. The reason for this is that such devices usually allow a diagnosis to be made without contact, that is to say in particular in a pain-free manner, and, in addition, often also provide optical images with which any necessary therapeutic measures can be communicated to the patient graphically and hence more clearly. For example, so-called intraoral cameras are used in dentistry, which cameras include a handpiece, the front end region of which is introduced into the mouth of a patient. In that end region there is generally a light-entry or viewing window for the camera lens, from which the image of the object to be examined is transmitted to an acquisition device, for example a CCD chip.

Such an intraoral camera can further be extended to a system for the transillumination of teeth, as is known inter alia from DE 10 2006 041 020 A1 of the applicant. In that system, the tooth to be examined is irradiated with light within a specific wavelength range, an optical image of the tooth illuminated by the examination radiation then being acquired and evaluated. Because carious areas in the tooth scatter the light differently than healthy dental tissue, such areas can be identified when the tooth is observed with the aid of a camera, it even being possible, if the system is suitably configured, to obtain a more reliable caries diagnosis than is the case with a conventional X-ray examination.

A further system for the transillumination of teeth is also known from DE 10 2009 013 615 A1 of the applicant. In that system, in particular the irradiation means, with the aid of which the light is directed at the tooth to be examined, are arranged in a removable attachment. This has advantages firstly because the attachment can be cleaned and in particular disinfected separately from the remainder of the handpiece. Secondly—as also already described in the above-mentioned DE 10 2009 013 615 A1—it is possible to provide different attachments which differ with regard to the manner in which the light is coupled into the tooth and the tooth is then observed. Accordingly, different teeth can each be examined in the optimum manner.

Starting from that prior art, the object underlying the present invention is to provide a dental system for the transillumination of teeth which permits more convenient use of the system and thus ultimately optimization of the examination.

The object is achieved by a dental system having the features of claim 1. Advantageous further developments of the invention are the subject of the dependent claims.

The dental system according to the invention for the transillumination of teeth also uses a handpiece at the front end of which there is arranged a removable attachment which contains at least part of the irradiation means. According to the invention, means are present on the handpiece with the aid of which the attachment arranged on the handpiece is automatically detected.

According to the present invention, therefore, there is proposed a dental system for the transillumination of teeth which comprises an elongate handpiece with a light source for producing examination radiation, irradiation means for directing the examination radiation at a tooth to be examined, and means for acquiring an optical image of the tooth illuminated by the examination radiation, wherein there is arranged at the front end of the handpiece a removable attachment which contains at least part of the irradiation means, and wherein according to the invention the handpiece comprises means for detecting the attachment arranged on the handpiece. Preferably, various attachments are available for the handpiece, the detection means being configured to detect both the presence of an attachment and the type of attachment fitted.

The possibility of automatically detecting the presence of an attachment at the front end of the handpiece and optionally even identifying its type gives rise to various improvements in the handling of the system. For example, it is possible in particular for the system to include a control circuit which, in dependence on information provided by the means for detecting the attachment, controls the light source for producing the examination radiation and/or the means for acquiring the optical image. For example, accidental activation of the light source with no attachment fitted could thus be prevented. It would also be conceivable to adapt the light intensity to the particular attachment present in order, for example, to avoid exposure to excessively high radiation intensities. Likewise, the image acquisition means could be adapted to the particular attachment used in respect of their mode of operation.

Where different attachments are available, they will differ primarily with regard to the configuration of the irradiation means, so that different possibilities for illuminating and observing teeth are provided. However, it would also be conceivable for at least one of the attachments to have a further light source with the aid of which a colored reflected image in particular of a dental tooth surface can be recorded. Because the transillumination of a tooth is generally carried out with a comparatively narrow-band light source, reflected images of a tooth surface produced with such light are generally not very meaningful. By using an additional light source, which can be produced, for example, by a white light source or an RGB light source, further optical images of a tooth can be produced. The functional scope of the system can thus be extended so that it can also be used as a conventional intraoral camera. The corresponding change of attachment can in turn preferably be detected independently by the system, which leads to an automatic change of operating mode in which corresponding parameters, such as, for example, aperture, focus and/or focal length of the image acquisition means, are adjusted.

The means for detecting the attachment can be configured in various ways. Light barriers, in particular fork light barriers, microswitches or Hall sensors have been found to be preferred forms.

Another advantageous further development of the present invention relates to the problem that a wet object field or an object field wetted with saliva can lead to greater reflection, reflections and/or refractions, as a result of which the quality of the image acquired by the image recording unit is markedly reduced. In order to avoid this, there is present according to an advantageous further development of the invention a device with the aid of which an air stream is directed at a light-entry window of the handpiece. This air stream, which can be applied continuously or as required, effects drying of the object field, as a result of which the above-described disadvantages are avoided. The quality of the images and ultimately the reliability of the caries diagnosis are thereby markedly increased.

According to another advantageous further development of the invention, the means for acquiring the optical image are further so configured that they comprise an electronic image recording unit and an image evaluation unit. It is thereby possible in particular for the image evaluation unit to control the image recording unit in such a manner that signals of high intensity are attenuated and signals of low intensity are amplified. By means of this procedure, disruptive effects caused by undesirable reflections of the light can be avoided. This in turn contributes towards improving the quality of the images.

The invention is to be explained in greater detail hereinbelow with reference to the accompanying drawing, in which:

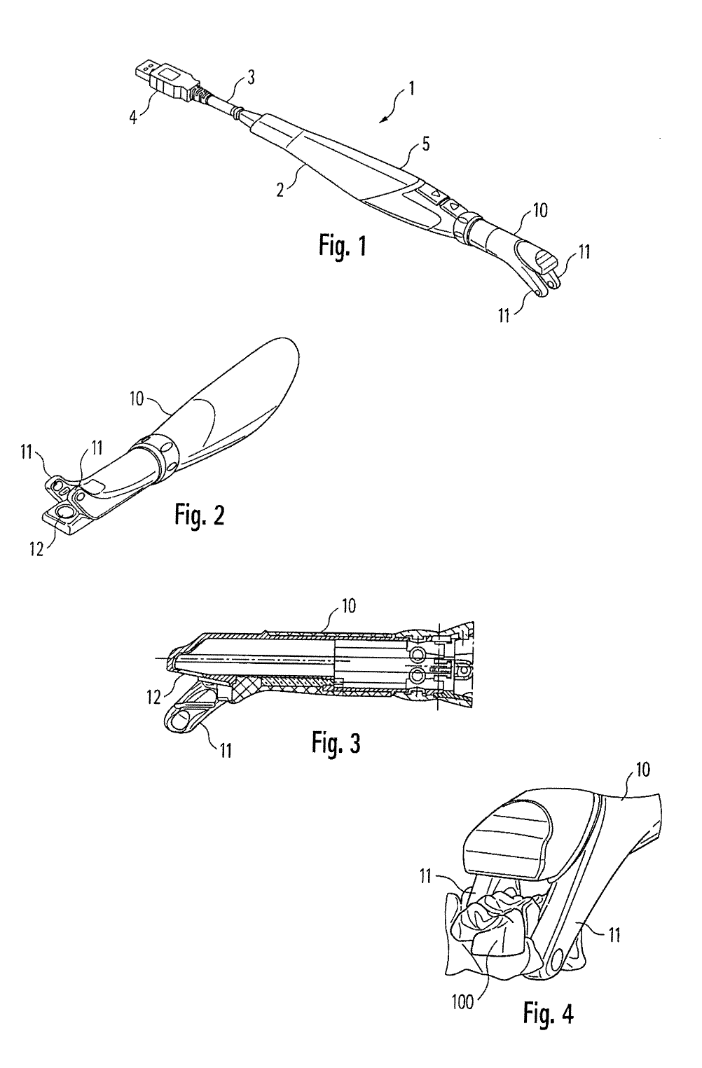

FIG. 1 shows a view of a dental system according to the invention for the transillumination of teeth;

FIG. 2 shows a first embodiment of an attachment used in the system according to the invention;

FIG. 3 shows a sectional view of the attachment of FIG. 2;

FIG. 4 shows in schematic form the use of the attachment of FIG. 2;

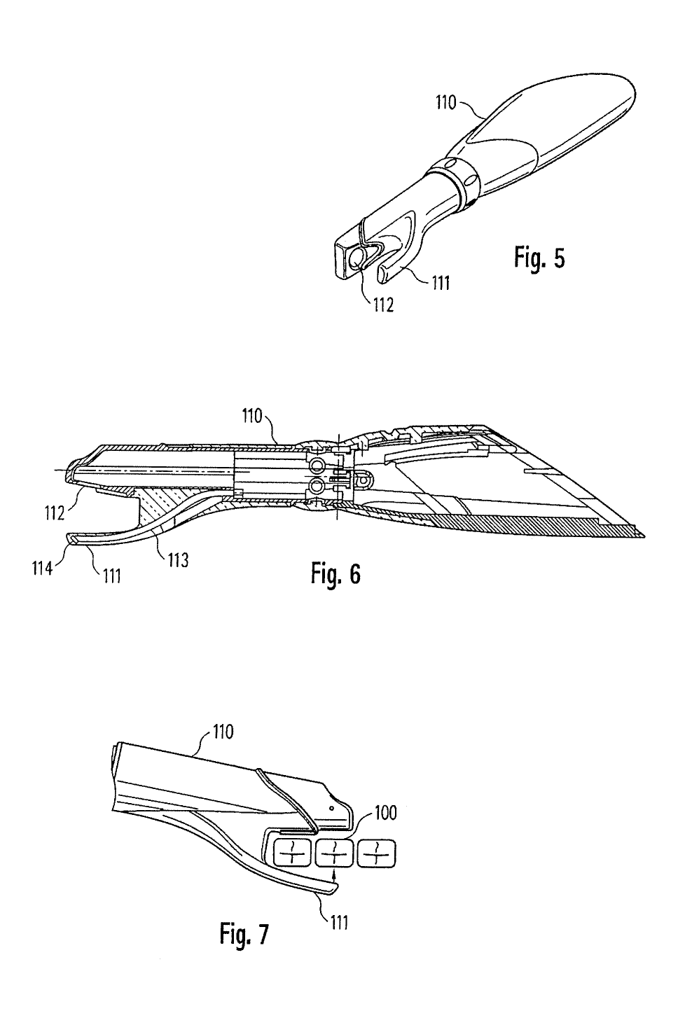

FIG. 5 shows a second embodiment of an attachment used in the system according to the invention;

FIG. 6 shows a sectional view of the attachment of FIG. 5; and

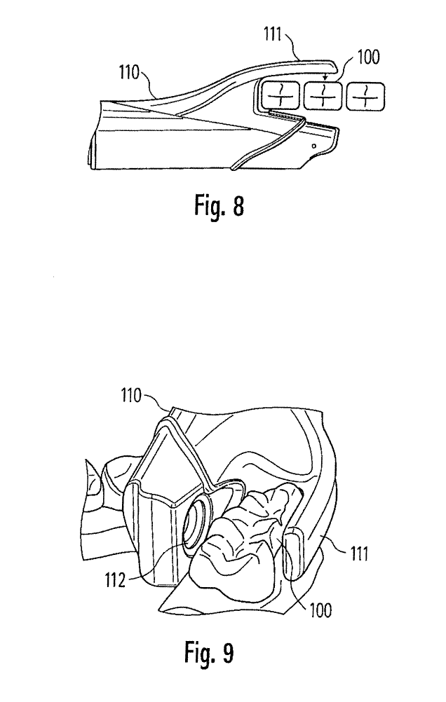

FIGS. 7 to 9 show views of the use of the second attachment of FIG. 5.

Before the dental device according to the invention is described in more detail, the method on which the examination method is based will first be explained briefly. The transillumination method is based on passing visible light through a tooth, that is to say transilluminating it. To that end, an examination radiation is generated with the aid of a light source and is directed at the tooth. The examination radiation is usually in a wavelength range of approximately from 550 μm to 790 μm, for example approximately 670 μm. The tissue of the tooth does not completely block the examination radiation but instead allows the light to pass through the tooth. The radiation is thereby partially scattered, carious regions in particular having a characteristic effect on the light. If the tooth transilluminated in that manner is viewed from different viewing directions, such carious regions can be detected because they appear slightly darker. In particular when images taken at different times are compared with one another, caries can thus be detected comparatively effectively and also in good time.

A so-called FOTI (fiber-optic transillumination) device based on this examination principle is shown in FIG. 1 and is denoted generally by the reference numeral 1. A fundamental component is a hand-held, elongate instrument 2 which is used to illuminate the tooth to be examined and also serves to acquire the optical image of the illuminated tooth. The data thereby obtained are transmitted via a cable 3, at the end of which there is a USB connector 4, to a central processing unit, in particular a PC (not shown). Here, the images produced are then displayed and the data are evaluated in order to detect caries. Of course, connection with the central unit could also be made by other means, but the USB connector 4 represents a preferred embodiment because the device 1 is thereby at the same time also supplied with power.

The hand-held instrument 2 consists firstly of an elongate handle 5 in which the fundamental electronic components of the device 1 are arranged. Those components are on the one hand the corresponding electronics for communication with the central unit via the USB connection, as well as means for image acquisition, recording and evaluation. It can be, for example, a CCD chip with the aid of which an optical image of the tooth is recorded.

Transfer of the optical image to the CCD chip located in the handle 5 is carried out with the aid of an attachment 10, which is removably arranged at the front end of the handle 5. The attachment 10, which is shown in greater detail in FIGS. 2 to 4, serves on the one hand to illuminate the tooth 100 to be examined, as is shown in FIG. 4. To that end there are used two lateral arms 11, through which light guides extend. At the two ends of the arms 11, the light is deflected laterally and coupled into the tooth 100. On the other hand, with the aid of the attachment 10, an image of the illuminated tooth 100 is acquired and passed by optical means to the image acquisition means, that is to say, for example, the CCD chip. To that end, the attachment 10 has at its front end a window 12 directed at the underside, which is coupled via further optical elements to the image acquisition means. For transmission of the image there are used primarily mirrors, prisms, lenses and the like.

FIGS. 5 to 9 show a further attachment 110 which could likewise be used in the device 1 according to the invention for caries diagnosis. In contrast to the attachment 10 discussed hereinbefore, this further attachment 110 has only a single arm 111, through which there extends a light guide 113 having a deflection element 114 at the front end. The end region having a light-entry window 112 is in turn arranged opposite the front end of the arm 111. When the attachment 110 is fitted to the handle 5 of the handpiece 2, a deflecting prism is located directly behind the entry window 112, with the aid of which deflecting prism the light incident on the tooth to be examined is directed to the image acquisition means.

The mutually opposing arrangement of the deflection element 114 and the light-entry window 112 has the result that, with the aid of the attachment 110, the tooth 100 to be examined can either be illuminated from the so-called buccal tooth surface and observed from the lingual tooth surface or alternatively illuminated from the lingual tooth surface and observed from the buccal tooth surface. Compared to embodiments conventional hitherto, such a changeover does not require the attachment to be replaced, which makes the operation much easier and represents a considerable time saving. Furthermore, it is also not necessary to remove the handpiece from the workspace, that is to say the oral cavity of the patient, and two-handed operation is not required in order to change the viewing direction. The attachment 110 is so arranged on the handpiece 2 that it cannot be twisted. Ergonomic handling is thereby achieved.

In the system according to the invention, therefore, at least two different attachments 10, 110 are used, which attachments permit optimum examination of a tooth for the particular application in question. Owing to the different configurations of the attachments 10, 110, however, it can be necessary suitably to adapt the light output and the sensitivity of the image acquisition means. For example, a higher light intensity may be necessary for the second attachment 110 because the tooth to be examined is here illuminated from only one side.

In order nevertheless to make handling of the system according to the invention simple and convenient, the fitted attachment 10 or 110 is detected automatically. That is to say, the handle 5 of the instrument 2 is provided with means by which it is detected whether there is an attachment at the front end of the handle 5 and—if yes—what type of attachment it is. Such means can in particular be in the form of a fork light barrier, in which case, when an attachment is fitted, part of the housing of the attachment, or a corresponding projection, enters the fork light barrier located on the handle. That part of the attachment can be opaque, colored, translucent or optically impermeable, identification of the attachment type then being made possible by a specific configuration of that region. Alternatively to that embodiment, automatic detection of the attachment could also be carried out with the aid of microswitches or Hall sensors with corresponding magnets.

Internally, the device 2 according to the invention then has a control unit which, on the basis of the information provided by the means for detecting the attachment, carries out corresponding control of the various components. In the simplest case, activation of the light source(s) is effected only when an attachment is fitted whereas, in the case where an attachment has not been detected, the light source(s) and any further functional units of the electronics are deactivated in order, for example, to avoid exposure to excessively high irradiation intensities. Adaptation of the light intensity to the type of attachment can—as already mentioned above—also be carried out thereby. Accordingly, the device independently detects which attachment has been fitted to the handle and carries out correspondingly suitable control of the further components of the system so that those adjustments do not have to be made manually by the user. Handling of the device is accordingly extremely simple despite the different possibilities.

It is further to be noted that the above-mentioned risk of exposure to excessively high irradiation intensities could also be avoided by providing an operating element which prevents the attachment from being removed when the light source is activated. Furthermore, use for detecting the presence of an attachment would also be expedient if only a single attachment was available, because incorrect operation, in particular activation of the light source(s) when no attachment is present, is to be avoided in that case too.

Another advantageous further development of the system according to the invention relates to the evaluation of the image information obtained by the CCD chip. In this connection it is to be noted that light which passes directly or by reflection from the illumination unit into the image recording unit, that is to say, for example, the CCD chip, without passing through human tissue has a markedly higher intensity than light which first passes through the tooth and is there attenuated by partial absorption. By means of a suitable configuration of the required image evaluation unit, it is possible to detect the spatial regions of the image recording unit which are illuminated with a markedly higher intensity. This can take place, for example, within the context of a so-called histogram analysis. The image evaluation unit can then control the image recording unit in such a manner that the associated brightness value of the over-illuminated regions is attenuated while at the same time those regions of the image recording unit which have a markedly lower intensity are amplified, so that the associated brightness signal is increased. The regions of the image recording unit thus modified in terms of their brightness value are then represented together in an image with a limited brightness range (for example 256 grey-scale values), as a result of which an image having a markedly increased dynamic range is ultimately obtained. In other words, the quality of the represented optical images, which is ultimately crucial for the reliability of the caries diagnosis, is markedly improved in this manner.

A further measure for improving the image quality consists in producing a sterilisable attachment with an integrated air channel. The background to this improvement is that a moist object field or an object field wetted with saliva leads to increased reflection, to reflections or refractions at air bubbles formed by saliva. Such effects necessarily lead to a reduced quality of the acquired image.

In order to avoid such effects, it is proposed according to a preferred embodiment to direct an air stream at the light-entry window of an attachment. By means of this air stream, the optical elements for recording images can be kept free of condensation and the object field can be dried. Preferably, the air is directed via the attachable attachment onto the optical element and optionally also onto the tooth to be examined. The air stream can be applied continuously. However, it would also be conceivable for the air stream to be switched on manually as required by the user of the device. Automatic switching on and off in conjunction with a sensor element which detects the presence of moisture on the object field to be recorded would also be conceivable.

It is important here that the air channel is guided through the attachment itself at least in its last section. It is thereby possible also to keep the channel free of germs within the context of the sterilization of the attachment. The air channel integrated into the attachment then communicates with an air supply line which passes through the handle of the handpiece.

The two attachment variants 10 and 110 shown both serve to obtain transillumination images of teeth. However, it would also be conceivable to provide a further attachment containing an additional light source which permits the recording of a colored reflected image of a dental tooth surface. The attachments shown in the figures are less suitable for that purpose because they only emit light with a very narrow spectral bandwidth. This is advantageous for the diagnosis of caries within the context of transillumination, but conventional optical images of a tooth surface cannot then be produced.

According to the proposed extension of the invention, therefore, a further attachment contains an additional light source, for example a white light source or an RGB light source. This is configured and arranged, in relation to the corresponding light-entry window of the attachment, in such a manner that it illuminates the region covered by the light-exit window directly and accordingly permits the production of a conventional reflected image. Ultimately, therefore, with the aid of this additional attachment, the system can also be used as a conventional intraoral camera. Where an RGB light source is used, the colored image can also be generated from a plurality of individual images in a downstream image evaluation device. In that case, therefore, the individual colors are activated in succession, the images obtained in each case being superposed and combined to form the image as a whole. The attachment used for that purpose has corresponding connections via which on the one hand power can be supplied to the light source or sources and on the other hand the light source(s) is/are controlled. It can again be provided that the fitting of this additional attachment is detected automatically by the device and a changeover to a different operating mode is accordingly initiated. When that change in operating mode is made, specific parameters of the image recording means, for example aperture, focus and/or focal point, can be adjusted accordingly.

Finally, in another advantageous further development of the invention, the means for image evaluation permit continuous recording, which ultimately allows a so-called panorama to be produced. In that case, the device is moved over the entire dental arch, image data or measured data continuously being acquired. In order to compress the measured data acquisition, it would be conceivable to combine the data of individual parts of the dental arch acquired at different time intervals in an overview. For example, by combining individual images of a live video stream it is possible to produce a single image of a larger part of the dental arch or of the complete dental arch. Such panoramas, which could hitherto be produced only with appropriately configured X-ray devices, allow necessary therapeutic measures to be explained to the patient in a particularly graphic and clear way.

Ultimately, therefore, a system for the transillumination of teeth is produced which can be handled by the user in a particularly simple and convenient manner. At the same time, however, it also allows images of very high quality to be produced. Ultimately, the possibility of the early detection of caries is markedly improved thereby.

1. Dental system for the transillumination of teeth, comprising:
an elongate handpiece with a light source for producing examination radiation,
irradiation means for directing the examination radiation at a tooth to be examined, and
means for acquiring an optical image of the tooth illuminated by the examination radiation,
wherein there is arranged at the front end of the handpiece a removable attachment that contains at least part of the irradiation means,
and
the handpiece has means for detecting the attachment arranged on the handpiece.

2. Dental system according to claim 1, wherein different attachments are available for the handpiece, the detection means being configured to detect both the presence of an attachment and the type of attachment.

3. Dental system according to claim 2, wherein the attachments differ with regard to the configuration of the irradiation means.

4. Dental system according to claim 2, wherein at least one of the attachments has an additional light source.

5. Dental system according to claim 4, wherein the additional light source is a white light source or an RGB light source.

6. Dental system according to claim 1, wherein the means for detecting the attachment has a light barrier, microswitches, or Hall sensors.

7. Dental system according to claim 1, comprising a control circuit which, in dependence on information provided by the means for detecting the attachment, controls the light source for producing the examination radiation and/or the means for acquiring the optical image.

8. Dental system according to claim 1, wherein the means for acquiring the optical image comprises a light-entry window located at a front end of the handpiece, the system having a device for directing an air stream onto the light-entry window.

9. Dental system according to claim 1, wherein the device for directing the air stream has an air channel extending through the attachment.

10. Dental system according to claim 1, wherein the means for acquiring the optical image comprises an electronic image recording unit and an image evaluation unit, the image recording unit being configured to control the image evaluation unit in such a manner that signals of high intensity are attenuated and signals of low intensity are amplified.
